(19) United States
(12) Patent Application Publication
(10) Pub. No.: US 2007/0212363 A1
(43) Pub. Date: Sep. 13, 2007

(54) Immunoglobulins with Potent and Broad Antiviral Activity

(75) Inventors: Dimiter S. Dimitrov, Frederick, MD (US); Mei-Yun Zhang, Frederick, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The Gov of USA, as Represented by the Secretary, Dept. of Health and Human Services, Rockville, MD (US)

(21) Appl. No.: 10/573,962

(22) PCT Filed: Sep. 29, 2004
(86) PCT No.: PCT/US04/31878

§ 371 Date: Oct. 2, 2006

Related U.S. Application Data
(60) Provisional application No. 60/506,946, filed on Sep. 29, 2003.

(51) Int. Cl. A61K 39/42; C07K 16/10; A61K 31/7072; A61K 38/13; A61K 38/20; C07H 21/04; C12P 21/06; A61K 39/21

(52) U.S. Cl. 424/160.1; 530/388.3; 435/69.1; 435/320.1; 435/326; 536/23.53; 514/11; 514/49; 424/208.1; 424/85.2; 424/85.4

(57) ABSTRACT

A (poly)peptide-Fc fusion molecule, such as an scFv-Fc fusion molecule comprising an scFv fragment and an Fc region from an antibody, related nucleic acids, vectors, and host cells, and a method of inhibiting a viral infection in a mammal, which method comprises administering to a mammal in need thereof the fusion molecule, wherein the fusion molecule binds to an epitope of a viral envelope protein that is inaccessible to whole immunoglobulin molecules due to molecular steric hindrance, or a nucleic acid, optionally in the form of a vector, encoding same, wherein the nucleic acid or vector is optionally contained within a host cell.

### FIELD OF THE INVENTION

The invention is directed to scFv-Fc fusion molecules and related nucleic acids, vectors, and host cells, and their use in the inhibition of a viral infection in a mammal.

### BACKGROUND OF THE INVENTION

The Human Immunodeficiency Virus (HIV) is the causative agent of Acquired Immunodeficiency Syndrome (AIDS). HIV type 1 (HIV-1) entry into host cells is initiated by the binding of the gp120 subunit of the viral envelope glycoprotein (Env) complex to the host cell receptor (CD4) (see, e.g., Dalgleish et al., Nature, 312, 763-767 (1984); and Klatzmann et al., Nature, 312, 767-768 (1984)). This interaction induces conformational changes in gp120 resulting in the exposure of a conserved high-affinity binding site for the co-receptor (i.e., the chemokine receptor CCR5 or CXCR4) (see, e.g., Sattentau et al., J. Exp. Med., 174, 407-415 (1991); Sattentau et al., J. Virol., 67, 7383-7393 (1993); Thali et al., J. Virol., 67, 3978-3988 (1993); Trkola et al., J. Virol., 70, 1100-1108 (1996); and Wu et al., Nature, 384, 179-183 (1996)).

Binding of Env to CD4 and either co-receptor initiates a series of conformational changes that lead to viral entry into the target cell. Therefore, efforts to develop a vaccine for the prevention and/or treatment of HIV infection have focused upon the development of neutralizing antibodies that specifically bind to Env. However, the extensive variation of Env in the numerous isolates of HIV so far identified presents a major obstacle in designing an effective immunogen for the isolation of antibodies with broadly neutralizing activity against multiple HIV isolates.

Neutralizing antibodies are believed to act, at least in part, by binding to the exposed Env surface and obstructing the initial interaction between a trimeric array of gp120 molecules on the virion surface and receptor molecules on the target cell (see, e.g., Parren et al., Adv. Immunol., 77, 195-262 (2001); Parren et al., J. Virol., 72, 3512-3519 (1998); and Ugolini et al., J. Exp. Med., 186, 1287-1298 (1997)). HIV-1 has evolved a number of strategies to evade recognition by neutralizing antibodies, particularly those directed to the conserved CD4 and co-receptor binding sites of Env. The extent of protection of these sites from antibody recognition is limited by the necessity to preserve the accessibility for receptor interaction. In the case of the binding site of CD4 (CD4bs), the following structural features have resulted: (i) CD4bs is partially obscured from antibody recognition by the V1/V2 loop and associated carbohydrate structures; (ii) the flanking residues are variable and modified by glycosylation; (iii) CD4bs is recessed to an extent that limits direct access by an antibody variable region; (iv) clusters of residues within the CD4bs that do not directly interact with CD4 are subject to variation among strains; (v) many gp120 residues interact with CD4 via main-chain atoms, allowing for variability in the corresponding amino acid side chains, and (vi) there is considerable conformational flexibility within the CD4-unbound state of gp120. Antibody binding, therefore, requires relatively large entropic decreases, thus conformationally masking the conserved CD4bs (see, e.g., Labrijn et al., J. Virol., 77(19), 10557-10565 (2003)).

The co-receptor binding site of gp120 is thought to be composed of a highly conserved element on the β19 strand and parts of the V3 loop (see, e.g., Rizzuto et al., AIDS Res. Hum. Retrovir., 16, 741-749 (2000); Rizzuto et al., Science, 280, 1949-1953 (1998); and Wyatt et al., Science, 280, 1884-1888 (1998)). These elements are masked by the V1/V2 variable loops in the CD4-unbound state and are largely unavailable for antibody binding (see, e.g., Trkola et al., Nature, 384, 184-187 (1996); and Wu et al., supra). Upon CD4 binding, conformational changes are induced, which include displacement of the V1/V2 stem-loop structure and consequent exposure of the co-receptor-binding site (see, e.g., Moore et al., J. Virol., 67, 6136-6151 (1993); Sattentau et al. (1993), supra; and Wyatt et al., J. Virol., 69, 5723-5733 (1995)). Binding studies with variable loop-deleted mutants suggest that CD4 induces additional rearrangement or stabilization of the gp120 bridging sheet near the β19 strand to form the final co-receptor-binding site (see, e.g., Wu et al., supra; and Wyatt et al. (1998), supra). Since the binding to CD4 occurs at the virus-cell interface, the exposed co-receptor binding site is optimally positioned for interaction with the co-receptor.

A highly conserved discontinuous structure on gp120 associated with the co-receptor binding site is recognized by monoclonal antibodies (mAbs) that bind better to gp120 upon ligation with CD4. These CD4-induced (CD41) antibodies, such as 17b and 48d, recognize a cluster of gp120 epitopes that are centered on the β19 strand and partially overlap the co-receptor binding site (see, e.g., Rizzuto et al. (2000), supra; Rizzuto et al. (1998), supra; Trkola et al. (1998), supra; and Wu et al., supra). Although such CD41 mAbs can neutralize some T-cell line-adapted HIV-1 strains, they are generally poorly neutralizing for primary isolates because their potency and related ability to suppress the generation of HIV-1 escape mutants are low. Recently, the antibody Fab fragment, X5, was isolated from a phage display library (see, e.g., International Patent Application WO 03/033666). Fab X5 is directed to a CD4i epitope and neutralizes a wide variety of primary isolates (see, e.g., Moulard et al., Proc. Natl. Acad. Sci. USA, 99, 6913-6918 (2002)), although the whole immunoglobulin G of X5, IgG X5, does not have a similar effect (see, e.g., Labrijn et al., supra). There remains a need for molecules with high neutralization activity and the ability to inhibit a broad range of HIV-1 primary isolates. The invention provides such a molecule, as well as methods of inhibiting a viral infection by administration of the molecule or a nucleic acid encoding the molecule. These and other objects and advantages of the invention, as well as additional inventive features, will be apparent from the description of the invention provided herein.

### BRIEF SUMMARY OF THE INVENTION

The invention is directed to a method of inhibiting a viral infection in a mammal, which method comprises administering to a mammal in need thereof an scFv-Fc fusion molecule comprising (a) a single chain-antibody variable region (scFv) fragment and (b) an Fc region of an antibody, wherein the scFv-Fc fusion molecule binds to an epitope of the viral envelope protein that is inaccessible to whole immunoglobulin molecules due to molecular steric hindrance, whereupon the viral infection is inhibited.

The invention also is directed to a method of inhibiting a viral infection in a mammal, which method comprises administering to a mammal in need thereof a nucleic acid molecule, optionally in the form of a vector, encoding an scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance, wherein the nucleic acid sequence or vector is optionally contained within a host cell, whereupon the viral infection is inhibited.

The invention provides an scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance.

The invention also provides a nucleic acid molecule encoding an scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance.

The invention also is directed to a vector comprising a nucleic acid molecule encoding scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance.

Additionally, the invention provides a host cell comprising a vector or nucleic acid molecule that encodes an scFv-Fc fusion molecule, wherein the scFv-Fc fusion molecule comprises (a) an scFv fragment and (b) an Fc region of an antibody, wherein the scFv-Fc fusion-molecule binds to an epitope of a viral envelope protein that is inaccessible to whole immunoglobulin molecules due to molecular steric hindrance.

### BRIEF DESCRIPTION OF THE DRAWINGS

FIG. 1 is a listing of the amino acid sequences described herein, wherein the sequences are presented from N-terminus to C-terminus from upper left to lower right in accordance with convention.

FIG. 2 is an SDS-PAGE of an scFv-Fc fusion molecule (i.e., m9Fc1 of SEQ ID NO:8) and a whole immunoglobulin molecule (human IgG1, see, e.g., PCT/US03/14292). Lane 1 is pre-stained protein rainbow markers; lane 2 is 2 μg of human IgG1; and lanes 3 and 4 are 2 μg of m9Fc1. Human IgG1 and m9Fc1 were expressed in 293 cells by transient transfection and purified from the supernatant by affinity purification using protein A column. The concentration of purified antibodies was determined by measuring the optimum density at 280 nm.

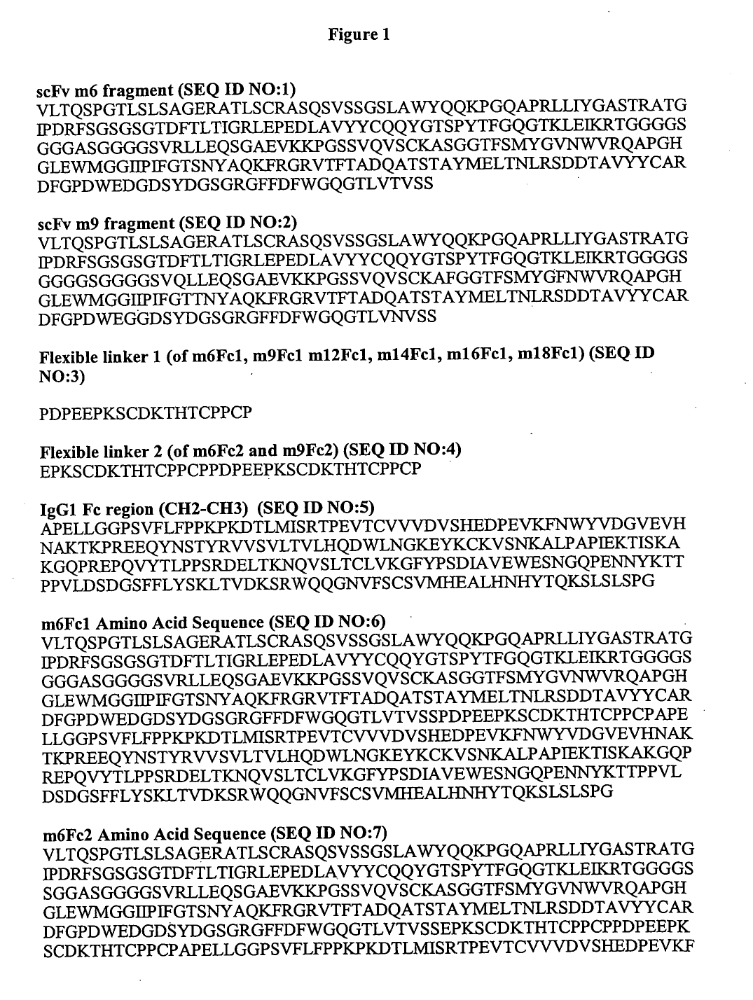

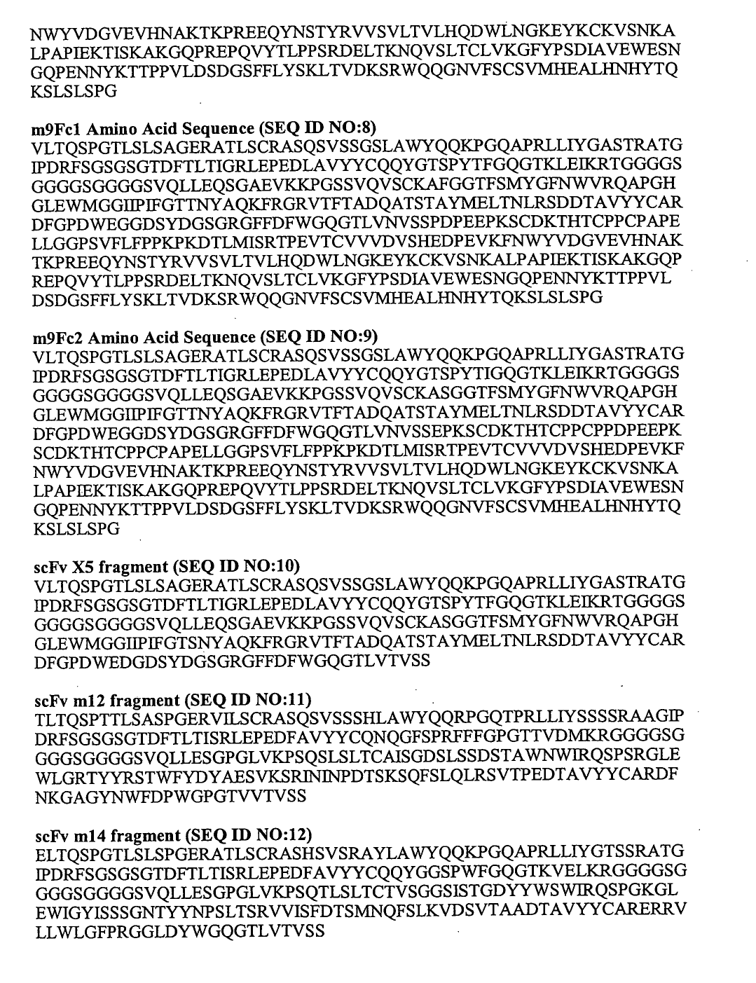

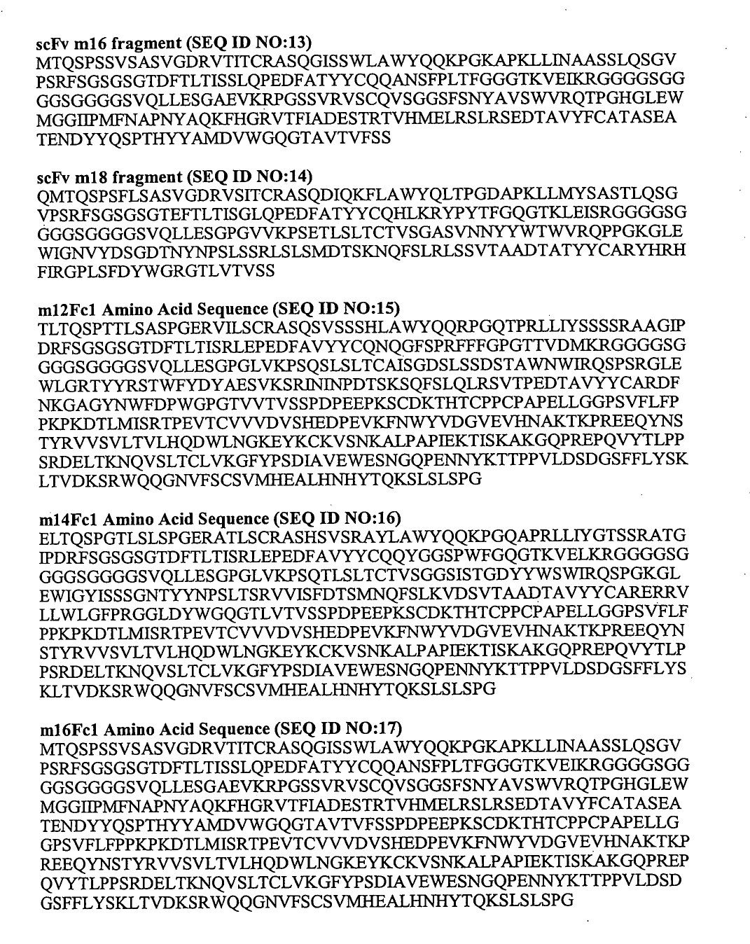

### DETAILED DESCRIPTION OF THE INVENTION

The invention is directed to a method of inhibiting a viral infection in a mammal, which method comprises administering to a mammal in need thereof a fusion molecule that comprises (a) a peptide or polypeptide comprising about 15 to about 300 amino acids, and (2) an Fc region of an antibody. The (poly) peptide-Fc fusion molecule binds to an epitope of the viral envelope protein that is inaccessible to whole immunoglobulin molecules due to molecular steric hindrance.

In one embodiment, the peptide or polypeptide is an scFv fragment. In this regard, the invention is directed to a method of inhibiting a viral infection in a mammal, which method comprises administering to a mammal in need thereof an scFv-Fc fusion molecule comprising (a) an scFv fragment and (b) an Fc region of an antibody, wherein the scFv-Fc fusion molecule binds to an epitope of the viral envelope protein that is inaccessible to whole immunoglobulin molecules due to molecular steric hindrance, whereupon the viral infection is inhibited.

The invention also is directed to a method of inhibiting a viral infection in a mammal, which method comprises administering to a mammal in need thereof a nucleic acid molecule, optionally in the form of a vector, encoding a (poly)peptide-Fc fusion molecule (e.g., an scFv-Fc fusion molecule comprising (a) an scFv fragment and (b) an Fc region of an antibody). The fusion molecule binds to an epitope of the viral envelope protein that is inaccessible to whole immunoglobulin molecules due to molecular steric hindrance, whereupon the viral infection is inhibited.

The viral infection to be inhibited by the administration of the (poly)peptide-Fc fusion molecule (e.g., an scFv-Fc fusion molecule), or nucleic acid molecule, encoding same, optionally in the form of vector, either one of which is contained within a host cell, can be any viral infection. In particular, the viral infection can be an infection by any virus normally trophic for a mammal, e.g., a human. For example, the infection can be an infection with a virus selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Lymphocytic choriomeningitis virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, SARS-CoV, Nipah virus, Hendra virus, HIV-1, and HIV-2. Preferably, the infection is with HIV-1 or HIV-2. Most preferably, the infection is with a HIV-1.

Inhibiting a viral infection refers to the inhibition in the onset of a viral infection, the inhibition of an increase in an existing viral infection, or a reduction in the severity of the viral infection. In this regard, one of ordinary skill in the art will appreciate that, while complete inhibition of the onset of a viral infection is desirable, any degree of inhibition of the onset of a viral infection, even for a period of time, is beneficial. Likewise, one of ordinary skill in the art will appreciate that, while elimination of viral infection is desirable, any degree of inhibition of an increase in an existing viral infection or any degree of a reduction of a viral infection is beneficial. Inhibition of a viral infection can be assayed by methods known in the art, such as by the assessment of viral load. Viral loads can be measured by methods that are known in the art, for example, using polymerase chain reaction assays to detect the presence of viral nucleic acid, or antibody assays to detect the presence of viral protein in a sample (e.g., blood) from a mammal. Alternatively, the number of CD4+ T cells in a viral-infected mammal can be measured. A treatment that inhibits an initial or further decrease in CD4+ T cells in a viral-infected mammal, or that results in an increase in the number of CD4+ T cells in a viral-infected mammal, is an efficacious treatment.

The mammal can be any mammal at risk for a viral infection or infected with a virus, such as a mouse, rat, rabbit, cat, dog, sheep, cow, horse, pig, or primate. Preferably, the mammal is a human.

The epitope of the viral envelope protein to which the (poly)peptide-Fc fusion molecule (e.g., scFv-Fc fusion molecule) binds can be any epitope that is inaccessible to larger molecules, such as whole immunoglobulin molecules (e.g., IgG1), due to molecular steric hindrance. One of ordinary skill in the art is familiar with assays to determine whether molecular steric hindrance affects the binding of two molecules (see, e.g., Labrijn et al., supra). The viral epitope is preferably a conserved viral epitope (i.e., the amino acid sequence of the epitope is shared by one or more strains and clades of the virus). Most preferably, the epitope is an epitope of HIV, such as an HIV envelope glycoprotein.

The (poly)peptide-Fc fusion molecule can be any (poly)peptide-Fc fusion molecule (e.g., an scFv-Fc fusion molecule) that can bind to an epitope of a viral envelope protein. The (poly)peptide-Fc fusion molecule can comprise any suitable peptide or polypeptide (e.g., an scFv fragment), any suitable Fc region, and, optionally, any suitable long flexible linker. When the (poly)peptide-Fc fusion molecule (e.g., scFv-Fc fusion molecule) comprises a linker, desirably the linker is positioned between the scFv fragment and Fc region.

The peptide or polypeptide of the (poly)peptide-Fc fusion molecule can be any suitable peptide or polypeptide that binds to an epitope of a viral envelope protein that is inaccessible to whole immunoglobulin molecules due to molecular steric hindrance. The peptide or polypeptide is sufficiently small in size, so as to avoid any steric, size, or orientation effects that prevent larger molecules (e.g., whole immunoglobulin molecules) from accessing the epitope of the viral envelope protein. Preferably, the peptide or polypeptide comprises about 15 to about 300 (e.g., about 25, about 50, about 75, about 100, about 125, about 150, about 175, about 200, about 225, about 250, about 275, or ranges thereof) amino acids. While the (poly)peptide can be any suitable peptide or polypeptide that has the above-described features, such as T-20 (see, e.g., Zhang et al., Clin. Pharmacol. Ther., 72(1), 10-19 (2002)), the (poly)peptide is preferably an scFv fragment.

The scFv fragment of the scFv-Fc fusion molecule can be any suitable scFv fragment. As known in the art, an scFv fragment comprises a heavy chain variable domain joined via a short linker peptide to a light chain variable domain, and is responsible for antigen binding. The scFv fragment to be used in the method of the invention is preferably broadly cross-reactive (e.g., can bind to a broad range of viral primary isolates from different strains and clades) with a high neutralization activity (e.g., typically with an IC₅₀ of less than 100 ug/ml). Preferably, the scFv fragment is an scFv fragment of m6 (SEQ ID NO:1) (see PCT/JUS03/14095), m9 (SEQ ID NO:2) (see PCT/US03/14095), X5 (SEQ ID NO:10) (see PCT/US03/14095), m12 (SEQ ID NO:11), m14 (SEQ ID NO:12), m16 (SEQ ID NO:13), m18 (SEQ ID NO:14), 17b (see, e.g., Choe et al., Cell, 114, 161-170 (2003)), 48d (see, e.g., Choe et al., supra), E51 (see, e.g., Choe et al., supra), 2F5 (see, e.g., Barbato et al., J. Mol. Biol., 330(5), 1101-15 (2003)), 4E10 (see, e.g., Zwick et al., J. Virol., 75(22), 10892-905 (2001)), or Z13 (see, e.g., Zwick et al., supra). Most preferably, the scFv fragment is scFv m6 (SEQ ID NO:1) or scFv m9 (SEQ ID NO:2). Alternatively, a variant of an aforementioned scFv fragment described above can be used. Desirably, the variant of the scFv fragment retains the ability to bind to the same epitope of the viral envelope protein. A variant of an scFv fragment can be obtained by any suitable method, including random and site-directed mutagenesis of the nucleic acid encoding the scFv fragment (see, e.g., Walder et al., Gene, 42, 133 (1986); Bauer et al., Gene, 37, 73 (1985); U.S. Pat. Nos. 4,518,584 and 4,732,462; and QuikChange Site-Directed Mutagenesis Kit (Stratagene, LaJolla, Calif.)) and sequential antigen panning (see, e.g., PCT/US03/14292). While a variant of the nucleic acid can be generated in vivo and then isolated and purified, alternatively, a variant of the nucleic acid can be synthesized. Various techniques used to synthesize nucleic acids are known in the art (see, e.g., Lemaitre et al., Proc. Natl. Acad. Sci., 84, 648-652 (1987)).

Additionally, a variant can be synthesized using peptide-synthesizing techniques known in the art (see, e.g., Bodansky, Principles of Peptide Synthesis, Springer-Verlag, Heidelberg, 1984). In particular, a (poly)peptide can be synthesized using the procedure of solid-phase synthesis (see, e.g., Merrifield, J. Am. Chem. Soc., 85, 2149-54 (1963); Barany et al., Int. J. Peptide Protein Res., 30, 705-739 (1987), and U.S. Pat. No. 5,424,398). If desired, a (poly)peptide can be synthesized with an automated peptide synthesizer. Removal of the t-butyloxycarbonyl (t-BOC) or 9-fluorenylmethyloxycarbonyl (Fmoc) amino acid blocking groups and separation of the (poly)peptide from the resin can be accomplished by, for example, acid treatment at reduced temperature. The (poly)peptide-containing mixture can then be extracted, for instance, with dimethyl ether, to remove non-peptidic organic compounds, and the synthesized (poly)peptide can be extracted from the resin powder (e.g., with about 25% w/v acetic acid). Following the synthesis of the (poly)peptide, further purification (e.g., using high performance liquid chromatography (HPLC)) optionally can be done in order to eliminate any incomplete (poly)peptides or free amino acids. Amino acid and/or HPLC analysis can be performed on the synthesized polypeptide to determine its identity. The (poly)peptide can be produced as part of a larger fusion protein, such as by the above-described methods or genetic means, or as part of a larger conjugate, such as through physical or chemical conjugation.

The ability of the variants of the above-described scFv fragments to bind to the same epitope of the viral envelope protein can be assessed by any suitable manner known in the art, such as by enzyme-linked immunosorbent assay (ELISA). The variant of the above-described scFv fragment includes molecules that have about 50% or more identity to the above-described scFv fragments. Preferably, the variant includes molecules that have 75% identity to the above-described scFv fragments. More preferably, the variant includes molecules that have 85% (e.g., about 90% or more, about 95% or more, about 96% or more, about 97% or more, about 98% or more, or about 99% or more) identity with the above-described scFv fragments. Ideally, the variant of the scFv fragment contains from 1 to about 40 (e.g., about 5, about 10, about 15, about 20, about 25, about 30, about 35, or ranges thereof) amino acid substitutions, deletions, inversions, and/or insertions thereof. More preferably, the variant of the above-described scFv fragments contains from 1 to about 20 amino acid substitutions, deletions, inversions, and/or insertions thereof. Most preferably, the variant of the scFv fragment contains from 1 to about 10 amino acid substitutions, deletions, inversions, and/or insertions thereof.

The substitutions, deletions, inversion, and/or insertions of the scFv fragment preferably occur in non-essential regions. The identification of essential and non-essential amino acids in the scFv fragment can be achieved by methods known in the art, such as by site-directed mutagenesis and AlaScan analysis (see, e.g., Moffison et al., Chem. Biol. 5(3), 302-307 (2001)). Essential amino acids have to be maintained or replaced by conservative substitutions in the variants of the scFv fragments, such that the scFv fragment maintains the ability to bind to an epitope of a viral envelope protein. Non-essential amino acids can be deleted, or replaced by a spacer or by conservative or non-conservative substitutions.

The variants can be obtained by substitution of any of the amino acids as present in the scFv fragment. As can be appreciated, there are positions in the sequence that are more tolerant to substitutions than others, and some substitutions can improve the binding activity of the native scFv fragment. The amino acids that are essential should either be identical to the amino acids present in the scFv fragment, or substituted by conservative substitutions. The amino acids that are nonessential can be identical to those in the scFv fragment, can be substituted by conservative or non-conservative substitutions, and/or can be deleted.

Conservative substitution refers to the replacement of an amino acid in the scFv fragment with a naturally or non-naturally occurring amino acid having similar steric properties. Where the side-chain of the amino acid to be replaced is either polar or hydrophobic, the conservative substitution should be with a naturally or non-naturally occurring amino acid that is also polar or hydrophobic (in addition to having the same steric properties as the side-chain of the replaced amino acid). When the native amino acid to be replaced is charged, the conservative substitution can be with a naturally or non-naturally occurring amino acid that is charged, or with a non-charged (polar, hydrophobic) amino acid that has the same steric properties as the side-chains of the replaced amino acid. For example, the replacement of arginine by glutamine, aspartate by asparagine, or glutamate by glutamine is considered to be a conservative substitution.

In order to further exemplify what is meant by conservative substitution, Groups A-F are listed below. The replacement of one member of the following groups by another member of the same group is considered to be a conservative substitution.

Group A includes leucine, isoleucine, valine, methionine, phenylalanine, serine, cysteine, threonine, and modified amino acids having the following side chains: ethyl, iso-butyl, —CH₂CH₂OH, —CH₂CH₂CH₂OH, —CH₂CHOHCH₃ and CH₂SCH₃.

Group B includes glycine, alanine, valine, serine, cysteine, threonine, and a modified amino acid having an ethyl side chain.

Group C includes phenylalanine, phenylglycine, tyrosine, tryptophan, cyclohexylmethyl, and modified amino residues having substituted benzyl or phenyl side chains.

Group D includes glutamic acid, aspartic acid, a substituted or unsubstituted aliphatic, aromatic or benzylic ester of glutamic or aspartic acid (e.g., methyl, ethyl, n-propyl, iso-propyl, cyclohexyl, benzyl, or substituted benzyl), glutamine, asparagine, CO—NH-alkylated glutamine or asparagine (e.g., methyl, ethyl, n-propyl, and iso-propyl), and modified amino acids having the side chain —(CH₂)₃COOH, an ester thereof (substituted or unsubstituted aliphatic, aromatic, or benzylic ester), an amide thereof, and a substituted or unsubstituted N-alkylated amide thereof.

Group E includes histidine, lysine, arginine, N-nitroarginine, p-cycloarginine, g-hydroxyarginine, N-amidinocitruiine, 2-amino guanidinobutanoic acid, homologs of lysine, homologs of arginine, and ornithine.

Group F includes serine, threonine, cysteine, and modified amino acids having C1-C5 straight or branched alkyl side chains substituted with —OH or —SH.

A non-conservative substitution is a substitution in which the substituting amino acid (naturally or non-naturally occurring) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, or isoleucine for glycine. Alternatively, a functional group can be added to the side chain, deleted from the side chain or exchanged with another functional group. Examples of nonconservative substitutions of this type include adding an amine, hydroxyl, or carboxylic acid to the aliphatic side chain of valine, leucine or isoleucine, or exchanging the carboxylic acid in the side chain of aspartic acid or glutamic acid with an amine or deleting the amine group in the side chain of lysine or ornithine.

For non-conservative substitutions, the side chain of the substituting amino acid can have significantly different steric and electronic properties from the functional group of the amino acid being substituted. Examples of such modifications include tryptophan for glycine, and lysine for aspartic acid.

The Fc region of the fusion molecule can be any suitable Fc region of an antibody. Preferably, the Fc region increases the stability, decreases the clearance time of the peptide or polypeptide (e.g., scFv fragment) from plasma and tissues, thereby enabling a minimum effective dose to be realized, and enhances an immune response to the virus. The Fc region of an antibody is limited in variability and is responsible for the biological effector function of the antibody (as opposed to the antigen-binding activity of the peptide or polypeptide (e.g., scFv fragment)), which is designed to bring about the destruction of the target recognized as foreign by the peptide or polypeptide (e.g., scFv fragment). The Fc portion varies between antibody classes (and subclasses) but is identical within that class. If the Fc region is a human Fc region, the Fc region is selected from the classes of IgA, IgD, IgE, IgG, and IgM. If the Fc region is an IgA or IgG Fc region, the subclass is selected from IgA1 and IgA2, or IgG1, IgG2, IgG3, and IgG4, respectively. Preferably, the Fc region is an Fc region of IgG, and most preferably, IgG1 (SEQ ID NO:5).

The peptide or polypeptide (e.g., scFv fragment) and Fc region of the fusion molecule optionally are joined together by a long flexible linker. The linker can be any suitable long flexible linker, such that the scFv fragment of the scFv-Fc fusion molecule can bind to the epitope of the viral envelope protein (i.e., the molecule is not excluded from binding by molecular steric hindrance). The linker can be any suitable length, but is preferably at least about 15 (e.g., at least about 20, at least about 25, at least about 30, at least about 35, at least about 40, at least about 45, at least about 50, or ranges thereof) amino acids in length. Preferably, the long flexible linker is an amino acid sequence that is naturally present in immunoglobulin molecules of the host, such that the presence of the linker would not result in an immune response against the linker sequence by the mammal. Preferably, the linker is the long flexible linker of SEQ ID NO:3 or SEQ ID NO:4.

The generation of the fusion molecules of the invention is within the ordinary skill in the art, and can comprise the use of restriction enzyme or recombinational cloning techniques (see, e.g., Gateway™ (Invitrogen) and U.S. Pat. No. 5,314,995).

As discussed above, the scFv-Fc fusion molecules encompassed by the invention comprise an scFv fragment, an Fc region, and, optionally, a flexible linker. Preferably, the scFv-Fc fusion molecules of the invention comprise SEQ ID NO:6, SEQ ID NO:7, SEQ ID NO:8, SEQ ID NO:9, SEQ ID NO:15, SEQ ID NO:16, SEQ ID NO:17, SEQ ID NO:18, or variants thereof, which retain the ability to bind to the same viral epitope. Further, any of these fusion molecules can be expressed with a suitable leader sequence, which leader sequence specifies how the fusion is trafficked through a cell expressing the leader-fusion polypeptide. A leader sequence among the preferred leader sequences is given in SEQ ID NO:19, but any suitable leader can be used.

Variants of the scFv-Fc fusion molecules can be obtained by any suitable method, including those methods discussed above. The ability of a variant to bind to the same epitope of the viral envelope protein can be assessed by any suitable manner known in the art, such as by ELISA. The variants of the above-described scFv-Fc fusion molecules include molecules that have about 90% or more percent identity (e.g., about 95% or more, about 96% or more, about 97% or more, about 98% or more, or about 99% or more) with the above-described scFv-Fc fusion molecules. Preferably, the variants of the scFv-Fc fusion molecules contain from 1 to about 50 (e.g., about 5, about 10, about 15, about 20, about 25, about 30, about 35, about 40, about 45, or ranges thereof) amino acid substitutions, deletions, inversions, and/or insertions thereof. More preferably, the variants contain from 1 to about 30 amino acid substitutions, deletions, inversions, and/or insertions thereof. Most preferably, the variants contain from 1 to about 20 amino acid substitutions, deletions, inversions, and/or insertions thereof. Ideally, the variants contain from 1 to about 10 amino acid substitutions, deletions, inversions, and/or insertions thereof. Preferably, the Fc region of the scFv-Fc fusion molecule and nucleotides encoding the same remain unchanged or are only slightly changed, such as by conservative or neutral amino acid substitution(s).

The (poly)peptide fusion molecule, such as an scFv-Fc fusion molecule (herein referred to as “fusion molecule”) preferably recognizes one or more strains of a virus. For example, if the viral infection to be inhibited by the administration of the fusion molecule is an HIV infection, the fusion molecule preferably recognizes an epitope of a viral envelope protein of HIV-1 and HIV-2. The fusion molecule also is preferably broadly cross-reactive (e.g., can bind to a wide range of isolates from different clades). For example, if the viral infection to be inhibited is an HIV infection, the fusion molecule preferably binds to an epitope of a viral envelope protein of clades A, B, C, D, E, EA, F, and/or G.

While the invention encompasses any fusion molecule that binds to an epitope of a viral envelope protein, the fusion molecule is preferably an antibody to HIV envelope glycoprotein (e.g., an HIV-1 envelope glycoprotein). The binding of the fusion molecule preferably is enhanced by the presence of CD4 and an HIV co-receptor (e.g., CXCR4 or CCR5). Preferably, the enhancement is exemplified by at least a two-fold increase in the binding affinity, such as a two-fold decrease in EC₅₀ as measured by ELISA.

The fusion molecule can be administered to a mammal as an amino acid molecule (i.e., as an scFv-Fc fusion molecule), as a nucleic acid encoding the fusion molecule, as a vector comprising the nucleic acid encoding the fusion molecule, or as a cell (e.g., a host cell) comprising any of the above. Vectors include nucleic acid vectors, such as naked DNA and plasmids, and viral vectors, such as retroviral vectors, parvovirus-based vectors (e.g., adenoviral-based vectors and adeno-associated virus (AAV)-based vectors), lentiviral vectors (e.g., Herpes simplex (HSV)-based vectors), and hybrid or chimeric viral vectors, such as an adenoviral backbone with lentiviral components (see, e.g., Zheng et al., Nat. Biotech., 18(2), 176-80 (2000); International Patent Application WO 98/22143; International Patent Application WO 98/46778; and International Patent Application WO 00/17376) and an adenoviral backbone with AAV components (see, e.g., Fisher et al., Hum. Gene Ther., 7, 2079-2087 (1996)). Vectors and vector construction are known in the art (see, e.g., Sambrook et al., Molecular Cloning: A Laboratory Manual, 2d edition, Cold Spring Harbor Laboratory, NY (1989); and Ausubel et al., Current Protocols in Molecular Biology, Green Publishing Associates and John Wiley & Sons, New York, N.Y. (1994)).

The vector can comprise any suitable promoter and other regulatory sequences (e.g., transcription and translation initiation and termination codons, which are specific to the type of host) to control the expression of the nucleic acid sequence encoding the fusion molecule. The promoter can be a native or normative promoter operably linked to the nucleic acid molecule described above. The selection of promoters, including various constitutive and regulatable promoters, is within the skill of an ordinary artisan. Examples of regulatable promoters include inducible, repressible, and tissue-specific promoters. Specific examples include viral promoters, such as adenoviral promoters and AAV promoters. Additionally, combining the nucleic acid described above with a promoter is within the skill in the art.

Host cells (e.g., isolated host cells) comprising the above-described fusion molecule or nucleic acid encoding the fusion molecule, optionally in the form of a vector, are also provided by the invention. Any suitable host cell can be used. Examples include host cells, such as E. coli (e.g., E. coli Tb-1, TG-2, DH5α, XL-Blue MRF′ (Stratagene), SA2821, and Y1090), Bacillus subtilis, Salmonella typhimurium, Serratia marcescens, Pseudomonas (e.g., P. aerugenosa), N. grassa, insect cells (e.g., Sf9, Ea4), yeast (S. cerevisiae) cells, and cells derived from a mammal, including human cell lines. Specific examples of suitable eukaryotic host cells include VERO, HeLa, 3T3, Chinese hamster ovary (CHO) cells, W138 BHK, COS-7, and MDCK cells. Alternatively, cells from a mammal, such as a human, to be treated in accordance with the methods described herein can be used as host cells. Methods of introducing vectors into isolated host cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J. 1, 841 (1982)). Desirably, the cell comprising the vector or nucleic acid sequence expresses the nucleic acid sequence, such that the nucleic acid sequence is transcribed and translated efficiently by the cell.

The nucleic acid molecules, vectors, cells, and fusion molecules can be administered to a mammal alone, or in combination with a pharmaceutically acceptable carrier. By pharmaceutically acceptable is meant a material that is not biologically or otherwise undesirable (i.e., the material can be administered to a mammal, along with the nucleic acid, vector, cell, or fusion molecule, without causing any undesirable biological effects or interacting in a deleterious manner with any of the other components of the pharmaceutical composition in which it is contained). The carrier is selected to minimize any degradation of the agent and to minimize any adverse side effects in the mammal, as would be well-known to one of ordinary skill in the art.

Suitable carriers and their formulations are described in Remington: The Science and Practice of Pharmacy (19th ed.) ed. A. R. Gennaro, Mack Publishing Company, Easton, Pa. (1995). Pharmaceutical carriers, include sterile water, saline, Ringer's solution, dextrose solution, and buffered solutions at physiological pH. Typically, an appropriate amount of a pharmaceutically acceptable salt is used in the formulation to render the formulation isotonic. The pH of the solution is preferably from about 5 to about 8 (e.g., about 5.5, about 6, about 6.5, about 7, about 7.5, and ranges thereof). More preferably, the pH is about 7 to about 7.5. Further carriers include sustained-release preparations, such as semipermeable matrices of solid hydrophobic polymers containing the fusion molecule, which matrices are in the form of shaped articles (e.g., films, liposomes, or microparticles). It will be apparent to those persons skilled in the art that certain carriers may be more preferable depending upon, for instance, the route of administration and concentration of composition being administered.

Compositions (e.g., pharmaceutical compositions) comprising the nucleic acid molecule, vector, cell, or fusion molecule can include carriers, thickeners, diluents, buffers, preservatives, surface active agents and the like. The compositions can also include one or more active ingredients, such as antimicrobial agents, anti-inflammatory agents, anesthetics, and the like.

The composition (e.g., pharmaceutical composition) comprising the nucleic acid molecule, vector, cell, or fusion molecule can be administered in any suitable manner depending on whether local or systemic treatment is desired, and on the area to be treated. Administration can be topically (including ophthalmically, vaginally, rectally, intranasally, transdermally, and the like), orally, by inhalation, or parenterally (including by intravenous drip or subcutaneous, intracavity, intraperitoneal, or intramuscular injection). Topical intranasal administration refers to the delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

If the composition is to be administered parenterally, the administration is generally by injection. Injectables can be prepared in conventional forms, either as liquid solutions or suspensions, solid forms suitable for solution of suspension in liquid prior to injection, or as emulsions. Additionally, parental administration can involve the preparation of a slow-release or sustained-release system, such that a constant dosage is maintained (see, e.g., U.S. Pat. No. 3,610,795). Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, and inert gases and the like.

Formulations for topical administration may include ointments, lotions, creams, gels, drops, suppositories, sprays, liquids, and powders. Conventional pharmaceutical carriers; aqueous, powder, or oily bases; thickeners; and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable.

Some of the compositions can potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lacetic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines.

The nucleic acid molecule, vector, or fusion molecules can be administered with a pharmaceutically acceptable carrier and can be delivered to the mammal's cells in vivo and/or ex vivo by a variety of mechanisms well-known in the art (e.g., uptake of naked DNA, liposome fusion, intramuscular injection of DNA via a gene gun, endocytosis, and the like).

Additionally, probiotic therapies are envisioned by the present invention. Viable host cells containing the nucleic acid or vector of the invention and expressing the fusion molecule can be used directly as the delivery vehicle for the fusion molecule to the desired site(s) in vivo. Preferred host cells for the delivery of the fusion molecule directly to desired site(s), such as, for example, to a selected body cavity, can comprise bacteria. More specifically, such host cells can comprise suitably engineered strain(s) of lactobacilli, enterococci, or other common bacteria, such as E. coli, normal strains of which are known to commonly populate body cavities. More specifically yet, such host cells can comprise one or more selected nonpathogenic strains of lactobacilli, such as those described by Andreu et al. (J. Infect. Dis., 171(5), 1237-43 (1995)), especially those having high adherence properties to epithelial cells (e.g., vaginal epithelial cells) and suitably transformed using the nucleic acid or vector of the invention.

If ex vivo methods are employed, cells or tissues can be removed and maintained outside the body according to standard protocols known in the art. The compositions can be introduced into the cells via any gene transfer mechanism, such as calcium phosphate mediated gene delivery, electroporation, microinjection, or proteoliposomes. The transduced cells then can be infused (e.g., with a pharmaceutically acceptable carrier) or homotopically transplanted back into the mammal per standard methods for the cell or tissue type. Standard methods are known for transplantation or infusion of various cells into a mammal.

The exact amount of the compositions required to treat a viral infection will vary from mammal to mammal, depending on the species, age, gender, weight, and general condition of the mammal, the nature of the virus, the existence and extent of viral infection, the particular fusion molecule, nucleic acid, vector, or cell used, the route of administration, and whether other drugs are included in the regimen. Thus, it is not possible to specify an exact amount for every composition. However, an appropriate amount can be determined by one of ordinary skill in the art using only routine experimentation given the teachings herein. Effective dosages and schedules for administering the nucleic acid molecules, vectors, cells, and fusion molecules of the invention can be determined empirically, and making such determinations is within the skill in the art. The dosage ranges for the administration of the compositions are those large enough to produce the desired effect; however, the dosage should not be so large as to cause adverse side effects, such as unwanted cross-reactions, anaphylacetic reactions, and the like. Dosage can vary, and can be administered in one or more (e.g., two or more, three or more, four or more, or five or more) doses daily, for one or more days. The composition can be administered before viral infection or immediately upon determination of viral infection and continuously administered until the virus is undetectable.

Guidance in selecting appropriate doses for antibodies, such as the fusion molecules of the invention, is found in the literature on therapeutic uses of antibodies (see, e.g., Handbook of Monoclonal Antibodies, Ferrone et al., eds., Noges Publications, Park Ridge, N.J., (1985); and Smith et al., Antibodies in Human Diagnosis and Therapy, Haber et al., eds., Raven Press, New York (1977)). A typical daily dosage of the fusion molecule used might range from about 1 μg/kg to up to 100 mg/kg of body weight or more per day, depending on the factors mentioned above. For example, the range can be from about 100 mg to one gram per dose. Nucleic acids, vectors, and host cells should be administered so as to result in comparable levels of production of fusion molecules.

The fusion molecule of the invention can be combined with other well-known therapies and prophylacetic vaccines already in use. The combination of the fusion molecule of the invention can generate an additive or a synergistic effect with current treatments. The fusion molecule of the invention can be combined with other HIV and AIDS therapies and vaccines, such as highly active antiretroviral therapy (HAART), azidothymidine (AZT), structured treatment interruptions of HAART, cytokine immune enhancement therapy (interleukin (IL)-2, IL-12, CD40L+IL-12, IL-7, and interferons (IFNs)), cell replacement therapy, recombinant viral vector vaccines, DNA vaccines, inactivated virus preparations, immunosuppressive agents, such as Cyclosporin A, and cyanovirin therapy (see, e.g., U.S. Pat. No. 6,015,876 and WO 03/072594). Such therapies can be administered in the manner already in use for the known treatment providing a therapeutic or prophylacetic effect (see, e.g., Silvestri et al. Immune Intervention in AIDS. In Immunology of Infectious Disease. H. E. Kauffman, A. Sher, and R. Ahmed eds., ASM Press. Washington D.C. 2002)).

The following examples further illustrate the invention but, of course, should not be construed as in any way limiting its scope.

### EXAMPLE 1

This example demonstrates that an scFv-Fc fusion molecule of the invention has greater neutralization activity than whole immunoglobulin molecules.

In this assay, single-round infectious molecular clones, produced by envelope complementation, were used. Plasmids containing the env genes of HIV-1 strains JR-FL, AD8, and IIIB and recombinant pseudovirions were produced as described in, for example, Labrijn et al., supra.

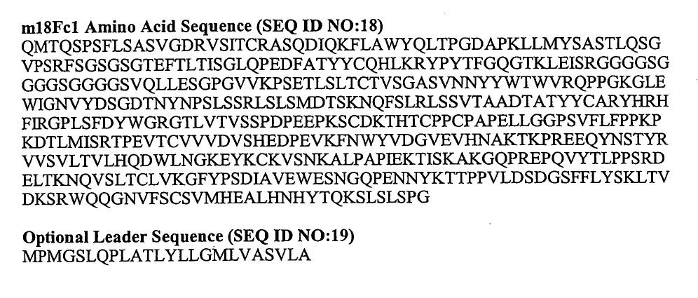

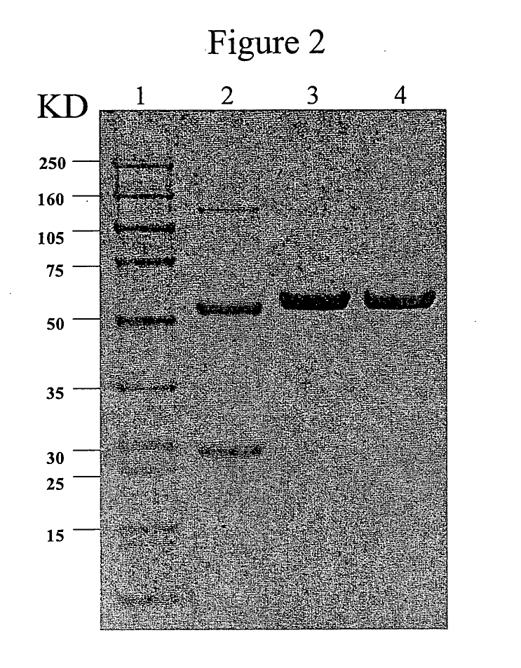

The degree of virus neutralization by antibody was achieved by measuring luciferase activity. Briefly, 2×10⁴ of neuroglioma U87 cells that had been transfected with CD4 and the chemokine receptors, CCR5 and CXCR4 (U87.CD4.CCR5.CXCR4 cells, Labrijn et al., supra) in 100 μl of medium (DMEM containing 15% FBS, 1 μg of puromycin/mL, 300 μg of G418/mL, glutamine, and penicillin-streptomycin) were added to microplate wells (96-well flat-bottom; Corning Inc., Corning, N.Y.) and incubated for 24 h at 37° C. in 5% CO₂. One hundred microliters of medium containing viral isolates, JR-FL, AD8, or IIIB, were mixed with various amounts of one of the following antibodies: scFv m9 (see, e.g., PCT/US03/14095), an scFv-Fc fusion molecule comprising scFv m9 (SEQ ID NO:8; FIG. 2), Fab X5 (see, e.g., International Patent Application WO 03/033666), Q4120 (Sigma, St. Louis, Mo.), or IgG1 X5 (see, e.g., Labrijn et al., supra). The mixtures were incubated for 1 h at 37° C., added to the cells, and incubated for a further 3 days. The wells were aspirated and washed once with PBS, and 60 μl of luciferase cell culture lysis reagent (Promega, Madison, Wis.) were added. The wells were scraped and the lysate was mixed by pipetting, 50 μl were transferred to a round-bottom plate (Corning), and the plate was centrifuged at 1,800×g for 10 min at 4° C. Twenty microliters were transferred to an opaque assay plate (Corning), and the luciferase activity was measured on a luminometer (EG&G Berthold LB 96V; Perkin Elmer, Gaithersburg, Md.) by using luciferase assay reagent (Promega).

Table 1 contains raw data representing the percentage of maximal infection for three HIV-1 isolates, JR-FL, AD8, and IIIB, in U87.CD4.CCR5.CXCR4 cells following administration of 100 μg/mL of scFv m9, an scFv-Fc fusion molecule comprising scFv m9 (m9-Fc), Fab X5, Q4120, or IgG1 X5. The level of infection was compared to CD4 negative cells (CD4-cells), which were assumed to be non-infectable (i.e. 100% inhibition).

TABLE 1
Infection in the presence of antibody
molecules for selected HIV-1 isolates

| | | | | | | CD4- |
|---|---|---|---|---|---|---|
| Isolate/Ab | scFv m9 | m9-Fc | IgG1 X5 | Fab X5 | Q4120 | cells |
| JR-FL | 20 ± 3 | 19 ± 1 | 72 ± 7 | 27 ± 9 | 12 ± 3 | 10 ± 3 |
| AD8 | 6.1 ± 1 | 5.5 ± 1 | 55 ± 12 | 8.7 ± 1 | 6.6 ± 1 | 5.8 ± 1 |
| IIIB | 12 ± 2 | 10 ± 1 | 28 ± 4 | 10 ± 1 | 14 ± 1 | 12 ± 1 |

Table 2 describes the neutralizing activity (percentage) in the presence of 100 μg/mL of m9, m9-Fc. IgG1 X5, Fab X5, or Q4120 in JR-FL, ADS, and IIIB in U87.CD4.CCR5.CXCR4 cells. The values of Table 2 were calculated based on the subtraction of background (CD4-cells).

TABLE 2
Neutralizing activity (percentage) in presence of antibody
molecules at 100 μg/mL for selected HIV-1 isolates

| | | | | | | CD4- |
|---|---|---|---|---|---|---|
| Isolate/Ab | scFv m9 | m9-Fc | IgG1 X5 | Fab X5 | Q4120 | cells |
| JR-FL | 90 | 91 | 38 | 83 | 98 | 100 |
| AD8 | 100 | 100 | 51 | 97 | 99 | 100 |
| IIIB | 100 | 100 | 84 | 100 | 98 | 100 |

As is apparent from the data set forth in Tables 1 and 2, the neutralization activity of scFv-Fc fusion molecules (e.g., m9-Fc) is much greater than that of whole immunoglobulin molecules (e.g., IgG1 X5).

### EXAMPLE 2

This example demonstrates the ability of fusion molecules comprising (a) a short peptide or polypeptide comprising about 15 to about 300 amino acids and (b) an Fc region of an antibody to bind to an epitope of a viral envelope protein that is inaccessible to whole immunoglobulin molecules due to molecular steric hindrance to inhibit a viral infection.

Enfuvirtide (T-20) is a 36 amino acid synthetic peptide that has been shown to block HIV entry into cells (see, e.g., Zhang et al., supra). A (T-20)-Fc fusion molecule comprising (a) T-20, (b) a long flexible linker (e.g., SEQ ID NO:3 or SEQ ID NO:4), and (c) an Fc region (e.g., SEQ ID NO:5) is generated. The neutralization activity of (T-20)-Fc fusion molecule is assayed in accordance with the method of Example 1. The neutralization activity of (T-20)-Fc fusion molecule is expected to be much greater than that of whole immunoglobulin molecules (e.g., IgG1 X5).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting a viral infection in a mammal, which method comprises administering to a mammal in need thereof an scFv-Fc fusion molecule comprising (a) a single chain-antibody variable region (scFv) fragment and (b) an Fc region of an antibody, wherein the scFv-Fc fusion molecule binds to an epitope of the viral envelope protein that is inaccessible to whole immunoglobulin molecules due to molecular steric hindrance, whereupon the viral infection is inhibited.

2. The method of claim 1, wherein the epitope is a conserved epitope.

3. The method of claim 1, wherein the whole immunoglobulin molecule is an IgG molecule.

4. The method of claim 1, wherein the mammal is a human and the viral infection is a human immunodeficiency virus (HIV) infection.

5. The method of claim 1, wherein the scFv-Fc fusion molecule is an antibody to HIV envelope glycoprotein.

6. The method of claim 1, wherein binding of the scFv-Fc fusion molecule is enhanced by the presence of CD4 and an HIV co-receptor.

7. The method of claim 6, wherein the co-receptor is CXCR4.

8. The method of claim 6, wherein the co-receptor is CCR5.

9. The method of claim 1, wherein the scFv-Fc fusion molecule recognizes one or more strains of HIV.

10. The method of claim 1, wherein the scFv-Fc fusion molecule can bind more than one lade of HIV.

11. The method of claim 1, wherein the scFv fragment comprises the amino acid sequence of SEQ ID NO:1, SEQ ID NO:2, SEQ ID NO:10, SEQ ID NO:11, SEQ ID NO:12, SEQ ID NO:13, SEQ ID NO:14, or a variant of any of the foregoing, wherein the variant retains the ability to bind to the same epitope.

12. The method of claim 1, wherein the scFv-Fc fusion molecule further comprises a flexible linker.

13. The method of claim 12, wherein the flexible linker comprises the amino acid sequence of SEQ ID NO:3 or SEQ ID NO:4.

14. The method of claim 1, wherein the Fc region comprises the amino acid sequence of SEQ ID NO:5.

15. The method of claim 1, wherein the fusion molecule comprises the amino acid sequence of SEQ ID NO:6, SEQ ID NO:7, SEQ ID NO:8, SEQ ID NO:9, SEQ ID NO:15, SEQ ID NO:16, SEQ ID NO:17, SEQ ID NO:18, or a variant of any of the foregoing, wherein the variant retains the ability to bind to the same epitope.

16. A method of inhibiting a viral infection in a mammal, which method comprises administering to a mammal in need thereof a nucleic acid molecule, optionally in the form of a vector, encoding an scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance, wherein the nucleic acid sequence or vector is optionally contained within a host cell, whereupon the viral infection is inhibited.

17. The method of claim 16, wherein the epitope is a conserved epitope.

18. The method of claim 16, wherein the whole immunoglobulin molecule is an IgG molecule.

19. The method of claim 16, wherein the mammal is a human and the viral infection is an HIV infection.

20. The method of claim 16, wherein the scFv-Fc fusion molecule is an antibody to HIV envelope glycoprotein.

21. The method of claim 16, wherein binding of the scFv-Fc fusion molecule to the viral epitope is enhanced by the presence of CD4 and an HIV co-receptor.

22. The method of claim 21, wherein the co-receptor is CXCR4.

23. The method of claim 21, wherein the co-receptor is CCR5.

24. The method of claim 16, wherein the scFv-Fc fusion molecule recognizes one or more strains of HIV.

25. The method of claim 16, wherein the scFv-Fc fusion molecule can bind more than one clade of HIV.

26. The method of claim 16, wherein the scFv fragment comprises the amino acid sequence of SEQ ID NO:1, SEQ ID NO:2, SEQ ID NO:10, SEQ ID NO:11, SEQ ID NO:12, SEQ ID NO:13, SEQ ID NO:14, or a variant of any of the foregoing, wherein the variant retains the ability to bind to the same epitope.

27. The method of claim 16, wherein the scFv-Fc fusion molecule further comprises a flexible linker.

28. The method of claim 27, wherein the flexible linker comprises the amino acid sequence of SEQ ID NO:3 or SEQ ID NO:4.

29. The method of claim 16, wherein the Fc region comprises the amino acid sequence of SEQ ID NO:5.

30. The method of claim 16, wherein the fusion molecule comprises the amino acid sequence of SEQ ID NO:6, SEQ ID NO:7, SEQ ID NO:8, SEQ ID NO:9, SEQ ID NO:15, SEQ ID NO:16, SEQ ID NO:17, SEQ ID NO:18, or a variant of any of the foregoing, wherein the variant retains the ability to bind to the same epitope.

31. An isolated or purified scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance.

32. The scFv-Fc fusion molecule of claim 31, wherein the epitope is a conserved epitope.

33. The scFv-Fc fusion molecule of claim 31, wherein the whole immunoglobulin molecule is an IgG molecule.

34. The scFv-Fc fusion molecule of claim 31, wherein the epitope is an HIV epitope.

35. The scFv-Fc fusion molecule of claim 31, wherein the epitope is an epitope from HIV envelope glycoprotein.

36. The scFv-Fc fusion molecule of claim 31, wherein binding of the scFv-Fc fusion molecule is enhanced by the presence of CD4 and an HIV co-receptor.

37. The scFv-Fc fusion molecule of claim 36, wherein the co-receptor is CXCR4.

38. The scFv-Fc fusion molecule of claim 36, wherein the co-receptor is CCR5.

39. The scFv-Fc fusion molecule of claim 31, wherein the scFv-Fc fusion molecule recognizes one or more strains of HIV.

40. The scFv-Fc fusion molecule of claim 31, wherein the scFv-Fc fusion molecule can bind more than one clade of HIV.

41. The scFv-Fc fusion molecule of claim 31, wherein the scFv fragment comprises the amino acid sequence of SEQ ID NO:1, SEQ ID NO:2, SEQ ID NO:10, SEQ ID NO:11, SEQ ID NO:12, SEQ ID NO:13, SEQ ID NO:14, or a variant of any of the foregoing, wherein the variant retains the ability to bind to the same epitope.

42. The scFv-Fc fusion molecule of claim 31, wherein the scFv-Fc fusion molecule further comprises a flexible linker.

43. The scFv-Fc fusion molecule of claim 31, wherein the flexible linker comprises the amino acid sequence of SEQ ID NO:3 or SEQ ID NO:4.

44. The scFv-Fc fusion molecule of claim 31, wherein the Fc region comprises the amino acid sequence of SEQ ID NO:5.

45. The scFv-Fc fusion molecule of claim 31, wherein the fusion molecule comprises the amino acid sequence of SEQ ID NO:6, SEQ ID NO:7, SEQ ID NO:8, SEQ ID NO:9, SEQ ID NO:15, SEQ ID NO:16, SEQ ID NO:17, SEQ ID NO:18, or a variant of any of the foregoing, wherein the variant retains the ability to bind to the same epitope.

46. An isolated or purified nucleic acid molecule encoding an scFv-Fc fusion molecule comprising (a) an scFv fragment and (b) an Fc region of an antibody, wherein the scFv-Fc fusion molecule binds to an epitope of a viral envelope protein that is inaccessible to whole immunoglobulin molecules due to molecular steric hindrance, and wherein the nucleic acid molecule is optionally in the form of a vector.

47. The nucleic acid molecule of claim 46, wherein the epitope is a conserved epitope.

48. The nucleic acid molecule of claim 46 or 17, wherein the whole immunoglobulin molecule is an IgG molecule.

49. The nucleic acid molecule of claim 46, wherein the epitope is an HIV epitope.

50. The nucleic acid molecule of claim 46, wherein the epitope is an epitope from HIV envelope glycoprotein.

51. The nucleic acid molecule of claim 46, wherein binding of the scFv-Fc fusion molecule is enhanced by the presence of CD4 and an HIV co-receptor.

52. The nucleic acid molecule of claim 51, wherein the co-receptor is CXCR4.

53. The nucleic acid molecule of claim 51, wherein the co-receptor is CCR5.

54. The nucleic acid molecule of claim 46, wherein the scFv-Fc fusion molecule recognizes one or more strains of HIV.

55. The nucleic acid molecule of claim 46, wherein the scFv-Fc fusion molecule can bind more than one clade of HIV.

56. The nucleic acid molecule of claim 46, wherein the scFv fragment comprises the amino acid sequence of SEQ ID NO:1, SEQ ID NO:2, SEQ ID NO:10, SEQ ID NO:11, SEQ ID NO:12, SEQ ID NO:13, SEQ ID NO:14, or a variant of any of the foregoing, wherein the variant retains the ability to bind to the same epitope.

57. The nucleic acid molecule of claim 46, wherein the scFv-Fc fusion molecule further comprises a flexible linker.

58. The nucleic acid molecule of claim 57, wherein the flexible linker comprises the amino acid sequence of SEQ ID NO:3 or SEQ ID NO:4.

59. The nucleic acid molecule of claim 46, wherein the Fc region comprises the amino acid sequence of SEQ ID NO:5.

60. The nucleic acid molecule of claim 46, wherein the fusion molecule comprises the amino acid sequence of SEQ ID NO:6, SEQ ID NO:7, SEQ ID NO:8, SEQ ID NO:9, SEQ ID NO:15, SEQ ID NO:16, SEQ ID NO:17, SEQ ID NO:18, or a variant of any of the foregoing, wherein the variant retains the ability to bind to the same epitope.

61. An isolated or purified host cell comprising a vector or nucleic acid molecule that encodes an scFv-Fc fusion molecule, wherein the scFv-Fc fusion molecule comprises (a) an scFv fragment and (b) an Fc region of an antibody, wherein the scFv-Fc fusion molecule binds to an epitope of a viral envelope protein that is inaccessible to whole immunoglobulin molecules due to molecular steric hindrance.

62. The host cell of claim 61, wherein the epitope is a conserved epitope.

63. The host cell of claim 61, wherein the whole immunoglobulin molecule is an IgG molecule.

64. The host cell of claim 61, wherein the epitope is an HIV epitope.

65. The host cell of claim 61, wherein the epitope is an epitope from HIV envelope glycoprotein.

66. The host cell of claim 61, wherein binding of the scFv-Fc fusion molecule is enhanced by the presence of CD4 and the HIV co-receptor.

67. The host cell of claim 66, wherein the co-receptor is CXCR4.

68. The host cell of claim 66, wherein the co-receptor is CCR5.

69. The host cell of claim 61, wherein the scFv-Fc fusion molecule recognizes one or more strains of HIV.

70. The host cell of claim 61, wherein the scFv-Fc fusion molecule can bind more than one clade of HIV.

71. The host cell of claim 61, wherein the scFv fragment comprises the amino acid sequence of SEQ ID NO:1, SEQ ID NO:2, SEQ ID NO:10, SEQ ID NO:11, SEQ ID NO:12, SEQ ID NO:13, SEQ ID NO:14, or a variant of any of the foregoing, wherein the variant retains the ability to bind to the same epitope.

72. The host cell of claim 61, wherein the scFv-Fc fusion molecule further comprises a flexible linker.

73. The host cell of claim 72, wherein the flexible linker comprises the amino acid sequence of SEQ ID NO:3 or SEQ ID NO:4.

74. The host cell of claim 61, wherein the Fc region comprises the amino acid sequence of SEQ ID NO:5.

75. The host cell of any of claim 61, wherein the fusion molecule comprises the amino acid sequence of SEQ ID NO:6, SEQ ID NO:7, SEQ ID NO:8, SEQ ID NO:9, SEQ ID NO:15, SEQ ID NO:16, SEQ ID NO:17, SEQ ID NO:18, or a variant of any of the foregoing, wherein the variant retains the ability to bind to the same epitope.

76. A composition comprising the scFv-Fc fusion molecule of claim 31 and a pharmaceutically acceptable carrier.

77. The composition of claim 76, wherein the composition further comprises an additional active agent.

78. The composition of claim 77, wherein the additional active agent is selected from the group consisting of azidothymidine (AZT), Cyclosporin A, inactivated virus, interleukin (IL)-2, IL-12, CD40 ligand and IL-12, IL-7, and an interferon.

79. A composition comprising the nucleic acid molecule of any of claim 46 and a pharmaceutically acceptable carrier.

80. The composition of claim 79, wherein the composition further comprises an additional active agent.

81. The composition of claim 80, wherein the additional active agent is selected from the group consisting of azidothymidine (AZT), Cyclosporin A, inactivated virus, interleukin (IL)-2, IL-12, CD40 ligand and IL-12, IL-7, and an interferon.

82. A composition comprising the host cell of claim 61 and a pharmaceutically acceptable carrier.

83. The composition of claim 82, wherein the composition further comprises an additional active agent.

84. The composition of claim 83, wherein the additional active agent is selected from the group consisting of azidothymidine (AZT), Cyclosporin A, inactivated virus, interleukin (IL)-2, IL-12, CD40 ligand and IL-12, IL-7, and an interferon.


SEQUENCE LISTING (19 sequences)

SEQ ID NO: 1 - protein, 254 aa, Artificial
  Synthetic
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTGGGGSGGGASGGGGSVRLLEQSGAEVKKPGSSVQVSCKASGGTFSMYGVNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSS

SEQ ID NO: 2 - protein, 254 aa, Artificial
  Synthetic
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTGGGGSGGGGSGGGGSVQLLEQSGAEVKKPGSSVQVSCKAFGGTFSMYGFNWVRQAPGHGLEWMGGIIPIFGTTNYAQKFRGRVTFTADQATSTAYMELTNLRSDDTAVYYCARDFGPDWEGGDSYDGSGRGFFDFWGQGTLVNVSS

SEQ ID NO: 3 - protein, 19 aa, Artificial
  Synthetic
PDPEEPKSCDKTHTCPPCP

SEQ ID NO: 4 - protein, 34 aa, Artificial
  Synthetic
EPKSCDKTHTCPPCPPDPEEPKSCDKTHTCPPCP

SEQ ID NO: 5 - protein, 216 aa, Artificial
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 489 aa, Artificial [sequence omitted]

SEQ ID NO: 7 - protein, 504 aa, Artificial
  Synthetic
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTGGGGSSGGASGGGGSVRLLEQSGAEVKKPGSSVQVSCKASGGTFSMYGVNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSSEPKSCDKTHTCPPCPPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 489 aa, Artificial
  Synthetic
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTGGGGSGGGGSGGGGSVQLLEQSGAEVKKPGSSVQVSCKAFGGTFSMYGFNWVRQAPGHGLEWMGGIIPIFGTTNYAQKFRGRVTFTADQATSTAYMELTNLRSDDTAVYYCARDFGPDWEGGDSYDGSGRGFFDFWGQGTLVNVSSPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 504 aa, Artificial
  Synthetic
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTIGQGTKLEIKRTGGGGSGGGGSGGGGSVQLLEQSGAEVKKPGSSVQVSCKASGGTFSMYGFNWVRQAPGHGLEWMGGIIPIFGTTNYAQKFRGRVTFTADQATSTAYMELTNLRSDDTAVYYCARDFGPDWEGGDSYDGSGRGFFDFWGQGTLVNVSSEPKSCDKTHTCPPCPPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 254 aa, Artificial
  Synthetic
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTGGGGSGGGGSGGGGSVQLLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSS

SEQ ID NO: 11 - protein, 247 aa, Artificial
  Synthetic
TLTQSPTTLSASPGERVILSCRASQSVSSSHLAWYQQRPGQTPRLLIYSSSSRAAGIPDRFSGSGSGTDFTLTISRLEPEDFAVYYCQNQGFSPRFFFGPGTTVDMKRGGGGSGGGGSGGGGSVQLLESGPGLVKPSQSLSLTCAISGDSLSSDSTAWNWIRQSPSRGLEWLGRTYYRSTWFYDYAESVKSRININPDTSKSQFSLQLRSVTPEDTAVYYCARDFNKGAGYNWFDPWGPGTVVTVSS

SEQ ID NO: 12 - protein, 247 aa, Artificial
  Synthetic
ELTQSPGTLSLSPGERATLSCRASHSVSRAYLAWYQQKPGQAPRLLIYGTSSRATGIPDRFSGSGSGTDFTLTISRLEPEDFAVYYCQQYGGSPWFGQGTKVELKRGGGGSGGGGSGGGGSVQLLESGPGLVKPSQTLSLTCTVSGGSISTGDYYWSWIRQSPGKGLEWIGYISSSGNTYYNPSLTSRVVISFDTSMNQFSLKVDSVTAADTAVYYCARERRVLLWLGFPRGGLDYWGQGTLVTVSS

SEQ ID NO: 13 - protein, 250 aa, Artificial
  Synthetic
MTQSPSSVSASVGDRVTITCRASQGISSWLAWYQQKPGKAPKLLINAASSLQSGVPSRFSGSGSGTDFTLTISSLQPEDFATYYCQQANSFPLTFGGGTKVEIKRGGGGSGGGGSGGGGSVQLLESGAEVKRPGSSVRVSCQVSGGSFSNYAVSWVRQTPGHGLEWMGGIIPMFNAPNYAQKFHGRVTFIADESTRTVHMELRSLRSEDTAVYFCATASEATENDYYQSPTHYYAMDVWGQGTAVTVFSS

SEQ ID NO: 14 - protein, 242 aa, Artificial
  Synthetic
QMTQSPSFLSASVGDRVSITCRASQDIQKFLAWYQLTPGDAPKLLMYSASTLQSGVPSRFSGSGSGTEFTLTISGLQPEDFATYYCQHLKRYPYTFGQGTKLEISRGGGGSGGGGSGGGGSVQLLESGPGVVKPSETLSLTCTVSGASVNNYYWTWVRQPPGKGLEWIGNVYDSGDTNYNPSLSSRLSLSMDTSKNQFSLRLSSVTAADTATYYCARYHRHFIRGPLSFDYWGRGTLVTVSS

SEQ ID NO: 15 - protein, 482 aa, Artificial
  Synthetic
TLTQSPTTLSASPGERVILSCRASQSVSSSHLAWYQQRPGQTPRLLIYSSSSRAAGIPDRFSGSGSGTDFTLTISRLEPEDFAVYYCQNQGFSPRFFFGPGTTVDMKRGGGGSGGGGSGGGGSVQLLESGPGLVKPSQSLSLTCAISGDSLSSDSTAWNWIRQSPSRGLEWLGRTYYRSTWFYDYAESVKSRININPDTSKSQFSLQLRSVTPEDTAVYYCARDFNKGAGYNWFDPWGPGTVVTVSSPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 482 aa, Artificial
  Synthetic
ELTQSPGTLSLSPGERATLSCRASHSVSRAYLAWYQQKPGQAPRLLIYGTSSRATGIPDRFSGSGSGTDFTLTISRLEPEDFAVYYCQQYGGSPWFGQGTKVELKRGGGGSGGGGSGGGGSVQLLESGPGLVKPSQTLSLTCTVSGGSISTGDYYWSWIRQSPGKGLEWIGYISSSGNTYYNPSLTSRVVISFDTSMNQFSLKVDSVTAADTAVYYCARERRVLLWLGFPRGGLDYWGQGTLVTVSSPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 485 aa, Artificial
  Synthetic
MTQSPSSVSASVGDRVTITCRASQGISSWLAWYQQKPGKAPKLLINAASSLQSGVPSRFSGSGSGTDFTLTISSLQPEDFATYYCQQANSFPLTFGGGTKVEIKRGGGGSGGGGSGGGGSVQLLESGAEVKRPGSSVRVSCQVSGGSFSNYAVSWVRQTPGHGLEWMGGIIPMFNAPNYAQKFHGRVTFIADESTRTVHMELRSLRSEDTAVYFCATASEATENDYYQSPTHYYAMDVWGQGTAVTVFSSPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 477 aa, Artificial
  Synthetic
QMTQSPSFLSASVGDRVSITCRASQDIQKFLAWYQLTPGDAPKLLMYSASTLQSGVPSRFSGSGSGTEFTLTISGLQPEDFATYYCQHLKRYPYTFGQGTKLEISRGGGGSGGGGSGGGGSVQLLESGPGVVKPSETLSLTCTVSGASVNNYYWTWVRQPPGKGLEWIGNVYDSGDTNYNPSLSSRLSLSMDTSKNQFSLRLSSVTAADTATYYCARYHRHFIRGPLSFDYWGRGTLVTVSSPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 24 aa, Artificial
  Synthetic
MPMGSLQPLATLYLLGMLVASVLA